(19) United States
(12) Patent Application Publication
(10) Pub. No.: US 2003/0055279 A1
(43) Pub. Date: Mar. 20, 2003

(54) Process for the preparation of benzyl carboxylates

(76) Inventors: Pieter Ooms, Krefeld (DE); Bernd-Ulrich Schenke, Bottrop (DE); Martin Sturmann, Leverkusen (DE)

Correspondence Address: BAYER CORPORATION, PATENT DEPARTMENT, 100 BAYER ROAD, PITTSBURGH, PA, 15205, US

(21) Appl. No.: 10/225,223

(22) Filed: Aug. 21, 2002

(30) Foreign Application Priority Data
Aug. 27, 2001 (DE) 10141830.2

(51) Int. Cl. C07C 253/30; C 07C 2 5/40; C 07C 2 5/61; C07C 67/24

(52) U.S. Cl. 558/414; 560/20; 560/240; 560/156

(57) ABSTRACT

Benzyl carboxylates can be prepared by reacting dibenzyl ethers with carboxylic acids and optionally carboxylic anhydrides in the presence of one or more, preferably one, acids applied to a support as catalyst.

### BACKGROUND

The present invention relates to a process for the preparation of benzyl carboxylates by reacting dibenzyl ethers with carboxylic acids and optionally carboxylic anhydrides in the molar ratio 1:1 to 1:50 at 10 to 200° C. and at pressures in the range from 0.1 to 50 bar in the presence of one or more, preferably one, heterogenized acids as catalyst.

Benzyl acetate, the main component of jasmine oil, is an important fragrance for the preparation of scent compositions and starting material for the preparation of fruit ethers.

The preparation of benzyl acetate by esterification of benzyl alcohol with acetic acid has been known for a long time.

Benzyl acetate can also be prepared by reacting benzyl chloride with alkali metal acetates, optionally in the presence of phase transfer reagents (Wang et al., Chem. Eng. Commun., 100, p.135 to 147 (1991)). A disadvantage is the formation of salts, which have to be disposed of and thus reduce the cost-efficiency of this process.

DD-A5-286 577 describes the preparation of benzyl acetate by reacting dibenzyl ether with acetic anhydride. Disadvantages are the drastic reaction conditions (300° C./20 MPa) and the only moderate yields.

The object was therefore to develop a process for the preparation of benzyl carboxylates starting from dibenzyl ethers which can be carried out under mild reaction conditions and which leads to good yields in a cost-effective manner.

### SUMMARY

The invention relates to a process for preparing a benzyl carboxylate of the formula

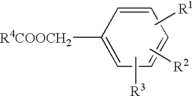

wherein

R¹ to R³ are identical or different and are C₁-C₆-alkyl, C₁-C₆-alkoxy, C₁-C₆-haloalkyl, C₁-C₆-haloalkoxy, CN, CO(C₁-C₆-alkyl), NO₂ or halogen and

R⁴ is hydrogen, C₁-C₆-alkyl, C₁-C₆-alkenyl, C₁-C₁₂-aryl, C₁-C₆-haloalkyl, C₁-C₆-haloalkenyl or C₁-C₁₂-haloaryl,

from dibenzyl ethers. The process comprises reacting (A) dibenzyl ethers of the formula

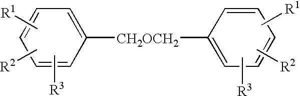

wherein R¹, R² and R³ have the meanings given above,

or mixtures of dibenzyl ethers and benzyl alcohols of the formula

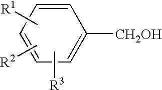

wherein R¹, R² and R³ have the meanings given above

with (B) carboxylic acids of the formula

R⁴COOH

wherein R⁴ has the meaning given above,

in the presence of a catalyst comprising at least one acid, wherein the acid is on a support.

### DESCRIPTION

We have found a process for the preparation of benzyl carboxylates of the formula

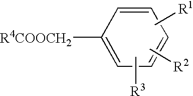

in which

R¹ to R³ are identical or different and are hydrogen, C₁-C₆-alkyl, C₁-C₆-alkoxy, C₁-C₆-haloalkyl, C₁-C₆-haloalkoxy, CN, CO(C₁-C₆-alkyl), NO₂ or halogen and

R⁴ is hydrogen, C₁-C₆-alkyl, C₁-C₆-alkenyl, C₁-C₁₂-aryl, C₁-C₆-haloalkyl, C₁-C₆-haloalkenyl or C₁-C₁₂-haloaryl,

from dibenzyl ethers, which is characterized in that dibenzyl ethers of the formula

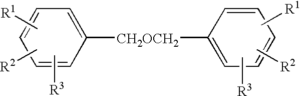

in which

R¹, R² and R³ have the meanings given above,

or mixtures of dibenzyl ethers and benzyl alcohols of the formula

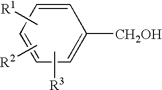

in which R¹, R² and R³ have the meanings given above are reacted with carboxylic acids of the formula

R⁴COOH

in which R⁴ has the meaning given above, in the presence of one or more acids applied to a support as catalyst.

The process according to the invention can be carried out in a cost-effective manner and under mild reaction conditions.

The radicals R¹ to R³ generally have the following meanings:

Alkyl generally means a straight-chain or branched hydrocarbon radical having 1 to 6, preferably 1 to 4, especially preferably 1 or 2, carbon atoms. Examples which may be mentioned are methyl, ethyl, propyl, isopropyl, butyl, isobutyl, pentyl, isopentyl, hexyl and isohexyl. Preference is given to methyl, ethyl, propyl, isopropyl, butyl, pentyl and hexyl, and particular preference is given to methyl and ethyl.

Alkoxy generally means a straight-chain or branched alkoxy radical having 1 to 6, preferably 1 to 4, especially preferably 1 or 2, carbon atoms. Examples which may be mentioned are methoxy, ethoxy, propoxy, isopropoxy, butoxy, isobutoxy, pentoxy, isopentoxy, hexoxy and isohexoxy. Preference is given to methoxy, ethoxy, propoxy, isopropoxy, butoxy, isobutoxy, pentoxy and hexoxy, and particular preference is given to methoxy and ethoxy.

Haloalkyl generally means a straight-chain or branched hydrocarbon radical having 1 to 6, preferably 1 to 4, particularly preferably 1 or 2, carbon atoms having 1 to 10, preferably 1 to 8, particularly preferably having 1 to 5, halogen atoms. Examples which may be mentioned are chloromethyl, fluoromethyl, difluoromethyl, trifluoromethyl, fluoroethyl, fluoropropyl and hexafluorobutyl. Preference is given to fluoromethyl, difluoromethyl, trifluoromethyl, fluoroethyl, fluoropropyl and hexafluorobutyl, and particular preference is given to fluoromethyl and trifluoromethyl.

Haloalkoxy generally means a straight-chain or branched alkoxy radical having 1 to 6, preferably 1 to 4, particularly preferably 1 or 2, carbon atoms having 1 to 10, preferably 1 to 8, particularly preferably having 1 to 5, halogen atoms. Examples which may be mentioned are chloromethoxy, fluoromethoxy, difluoromethoxy, trifluoromethoxy, fluoroethoxy, fluoropropoxy and hexafluorobutoxy. Preference is given to chloromethoxy, fluoromethoxy, trifluoromethoxy, fluoroethoxy, fluoropropoxy and hexafluorobutoxy, particular preference being given to fluoromethoxy and trifluoromethoxy.

Halogen generally means fluorine, chlorine, bromine and iodine, preferably fluorine, chlorine and bromine, in particular fluorine and chlorine.

Very particularly preferred substituents for R¹ to R³ are hydrogen, methyl, trifluoromethyl, methoxy, fluorine or chlorine.

According to the process of the invention, the following benzyl carboxylates can, for example, be prepared: benzyl formate, benzyl acetate, benzyl propionate, benzyl butyrate, benzyl pentanoate, benzyl hexanoate, benzyl heptanoate, benzyl octanoate, benzyl nonanoate, benzyl decanoate, benzyl undecanoate, benzyl dodecanoate, benzyl tridecanoate, benzyl tetradecanoate, benzyl pentadecanoate, benzyl hexadecanoate, benzyl heptadecanoate, benzyl octadecanoate, benzyl nonadecanoate, benzyl 2-hydroxybenzoate, benzyl 3-hydroxybenzoate, benzyl 4-hydroxybenzoate, benzyl 3-chloro-2-hydroxybenzoate, benzyl 4-chloro-2-hydroxybenzoate, benzyl 5-chloro-2-hydroxybenzoate, benzyl 6-chloro-2-hydroxybenzoate, benzyl 3-bromo-2-hydroxybenzoate, benzyl 3-fluoro-2-hydroxybenzoate, benzyl 4-fluoro-2-hydroxybenzoate, benzyl 2-fluoro-3-hydroxybenzoate, benzyl 2-fluoro-4-hydroxybenzoate, benzyl 3-fluoro-2-hydroxybenzoate, benzyl 2-fluoro-5-hydroxybenzoate, benzyl 2-fluoro-6-hydroxybenzoate, benzyl 2-hydroxy-3-methylbenzoate, benzyl 2-hydroxy-4-methylbenzoate, benzyl 3-hydroxy-2-methylbenzoate, benzyl 4-hydroxy-2-methylbenzoate, benzyl 2-fluoro-6-hydroxy-4-methoxybenzoate, benzyl 3-trifluoromethyl-2-hydroxybenzoate, benzyl 4-trifluoromethyl-2-hydroxybenzoate, benzyl 2-trifluoromethyl-3-hydroxybenzoate, benzyl 2-fluoroethyl-4-hydroxybenzoate and benzyl 4-fluorobutyl-2-hydroxybenzoate.

The dibenzyl ether used in the process according to the invention is an unsubstituted or substituted dibenzyl ether. Particular preference is given to using unsubstituted dibenzyl ether.

In the process according to the invention, it is possible to use dibenzyl ether or dibenzyl ether/benzyl alcohol mixtures as are produced, for example, during the preparation of benzyl alcohol from benzyl chloride. The content of dibenzyl ether may be from about 50 to about 100%, preferably from about 60 to about 99%, particularly preferably from about 70 to about 98%.

The carboxylic acids used in the process according to the invention are straight-chain and branched, saturated and unsaturated alkyl-, aralkyl- and arylcarboxylic acids having 1 to 50 carbon atoms, such as formic acid, acetic acid, propionic acid, butyric acid, isobutyric acid, valeric acid, isovaleric acid, caproic acid, heptanoic acid, caprylic acid, nonanoic acid, capric acid, undecanoic acid, lauric acid, tridecanoic acid, myristic acid, palmitic acid, stearic acid, oleic acid, linoleic acid, chloroacetic acid, linolenic acid, acrylic acid, methacrylic acid, cinnamic acid, phenylacetic acid, benzoic acid or salicylic acid. Preference is given to carboxylic acids having 2 to 30 carbon atoms, particularly preferably 2 to 10 carbon atoms. Very particularly preferred carboxylic acids are formic acid, acetic acid, chloroacetic acid, propionic acid and hexanoic acid. The process according to the invention is preferably carried out with removal of the water formed. It is appropriate to remove the water by distillation or by passing through an inert gas, such as nitrogen. Preference is given to removing the water formed using dehydrating agents, for example zeolites, aluminium oxides or clay earths. Particular preference is given to removing the water formed by carrying out the reaction in the presence of the corresponding anhydride of the carboxylic acid used as dehydrating agent. Very particularly preferred anhydrides are acetic anhydride, chloroacetic anhydride, propionic anhydride and benzoic anhydride.

In the process according to the invention, preference is given to using from about 2 to about 50 equivalents of carboxylic acid, preferably from about 3 to about 30 equivalents, particularly preferably from about 4 to about 20 equivalents, based on dibenzyl ether.

If the process according to the invention is carried out in the presence of the corresponding anhydride of the carboxylic acid used, then preference is given to using from about 0.1 to about 10 equivalents of anhydride, preferably from about 0.5 to about 7.5 equivalents, particularly preferably from about 1 to about 5 equivalents, based on dibenzyl ether. Since one molecule of anhydride used reacts with the uptake of water to give 2 molecules of carboxylic acid, it is possible to use smaller amounts of carboxylic acid in the process according to the invention. From about 1 to about 25 equivalents of carboxylic acid, preferably from about 1.5 to about 15 equivalents, particularly preferably from about 2 to about 10 equivalents, of carboxylic acid, based on dibenzyl ether, are then preferably used.

Suitable catalysts for the process according to the invention are inorganic acids, e.g., sulfur trioxide, sulfuric acid, hydrogen chloride, hydrogen bromide, hydrogen iodide, hydrofluoric acid, perchloric acid, chlorosulfonic acid or phosphoric acid, organic acids, e.g., trifluoroacetic acid, methanesulfonic acid, ethanesulfonic acid, benzenesulfonic acid, 4-toluenesulfonic acid or trifluoromethanesulfonic acid and Lewis acids, e.g., boron trifluoride, aluminium chloride, aluminium bromide, aluminium iodide, zinc chloride, tin chloride, titanium chloride or zirconium chloride, applied to one or more, preferably one, supports.

Preference is given to sulfur trioxide, sulfuric acid, trifluoromethanesulfonic acid, 4-toluenesulfonic acid and boron trifluoride, applied to supports, particularly preferably sulfur trioxide, sulfuric acid, trifluoromethanesulfonic acid and boron trifluoride, applied to a support.

Suitable supports for the process according to the invention are oxides or sulfates of elements of groups II A (group 2 according to IUPAC), for example, magnesium, calcium or barium, III B (group 3 according to IUPAC), e.g., scandium, yttrium or lanthanum, IV B (group 4 according to IUPAC), e.g., titanium, zirconium or hafnium, V B (group 5 according to IUPAC), for example, niobium or tantalum, VII B (group 7 according to IUPAC), e.g., manganese, VIII (groups 8, 9 and 10 according to IUPAC), e.g., iron or nickel, III A (group 13 according to IUPAC), for example Al and IV A (group 14 according to IUPAC), for example silicon, germanium, tin or lead and carbon.

Examples which may be mentioned are CaO, MgO, ZrO2, TiO₂, HfO₂, SnO₂, Al₂O₃, SiO₂, Al₂O₃.SiO₂ (alumosilicates such as zeolites or phyllosilicates), Nb₂O₅, Ta₂O₅, Fe₂O₃, LaSO₄ or CaSO₄ and activated carbons.

Preference is given to CaO, MgO, ZrO₂, TiO₂, HfO₂, SnO₂, Al₂O₃.SiO₂, Al₂O₃, SiO₂, Nb₂O₅, Ta₂O₅, Fe₂O₃, LaSO₄ or CaSO₄, and particular preference to CaO, MgO, SnO₂, ZrO₂, TiO₂, HfO₂, Al₂O₃, SiO₂, Al₂O₃.SiO₂, Nb₂O₅ and Ta₂O₅.

Very particularly preferred catalysts are sulfated oxides (superacid), such as SO₃ on CaO, MgO, ZrO₂, TiO₂, HfO₂, SnO₂, Al₂O₃, SiO₂, Al₂O₃, SiO₂, Nb₂O₅, Ta₂O₅ or Fe₂O₃.

Methods for the preparation are well known and described, for example, in Applied Catalysis A, 146, 1996, p. 3 to 32, Catalysis Today 20, p. 219 to 256 (1994) and WO 00/64849.

The acids or hydrates thereof can be used applied to a support, optionally calcined, as heterogeneous catalyst.

The catalysts can be used, for example, as powders or moldings and be separated off after the reaction by, for example, filtration, sedimentation or centrifugation.

In cases where the arrangement is in the form of a fixed bed, the acids are preferably applied to a support and used as moldings, e.g. as beads, cylinders, rods, hollow cylinders, rings etc.

These heterogenized acids are, where necessary, dried by heat, optionally under reduced pressure, optionally by washing with hydrophilic organic liquids, e.g., the carboxylic acid used or the carboxylic anhydride used, or optionally by azeotropic distillation with organic liquids such as toluene, xylene or methylene chloride.

The supported acids are used, when working with a suspended catalyst in stirred vessels, in amounts ranging from about 0.1 to about 100% by weight, preferably from about 0.5 to about 90% by weight, and particularly preferably from about 1.0 to about 80% by weight, based on dibenzyl ether.

In the case of a continuous procedure in countercurrent or cocurrent or in the trickle phase over a fixed-bed catalyst, space velocities of from about 0.05 g to about 5000 g of dibenzyl ether per liter of immobilized acid are used, preferably from about 0.1 to about 4000 g/l.h and particularly preferably from about 1.0 to about 3000 g/l.h.

The process according to the invention is preferably carried out with intensive thorough mixing of the reactants. Intensive thorough mixing can be achieved in various ways known to the person skilled in the art, for example by stirrers, nozzles, baffles, static mixers, pumps, turbulent flow into narrow tubes or by ultrasound.

Such devices are described in more detail in Ullmann's Encyclopedia of Industrial Chemistry, 5th Edition, Volume B, Unit Operations, Sections 25, 26, B4 pp. 569 to 570, Verlag Chemie, Weinheim 1988.

A preferred embodiment of the process according to the invention involves adding dibenzyl ether to a mixture or suspension of the supported acid and carboxylic acid and/or carboxylic anhydride and, after the reaction is complete, separating off the catalyst by, for example, filtration or centrifugation.

A further preferred embodiment is the cocurrent process in which dibenzyl ether and carboxylic acid and/or carboxylic anhydride are applied in cocurrent, for example, from the top downwards onto a catalyst bed arranged in a tube, and benzyl carboxylates are drawn off at the foot of the tube.

In a further preferred embodiment of the process according to the invention, this is carried out in the trickle phase and the supported acid is in the form of a fixed-bed catalyst. The catalyst bed is preferably located in a vertical tubular reactor which preferably contains intermediate plates to better distribute the stream of liquid and to better wet the catalyst bed.

Both in the case of the suspended catalyst and also in the fixed bed process variant, work-up may involve adding a water-immiscible solvent, preferably toluene, to the reaction products. After the organic phase, which comprises the crude benzyl carboxylate, has been separated off, it can be further purified, for example by distillation.

The process according to the invention can be carried out batchwise, continuously or semicontinuously.

The temperature at which the process according to the invention is carried out is preferably from about 15 to about 200° C., particularly preferably from about 25 to about 190° C., very particularly preferably from about 30 to about 180° C.

If the reaction is carried out above 115° C., it is necessary to work under increased pressure corresponding to the vapour pressure. The gauge pressure required is then at least equal to the vapour pressure of the reaction mixture. It may be up to about 50 bar, preferably up to 25 bar.

Where appropriate, the process according to the invention can be carried out under a customary protective gas, e.g., nitrogen, helium or argon.

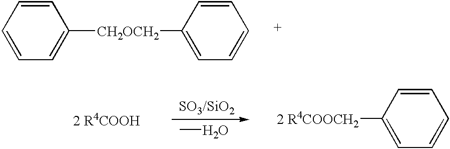

The process according to the invention gives benzyl carboxylates in good yields with a high conversion and good selectivity. The process according to the invention can be carried out simply without high expenditure on apparatus.

The invention is further illustrated but is not intended to be limited by the following examples in which all parts and percentages are by weight unless otherwise specified.

### EXAMPLES

The percentages given in the examples below are based on the weight.

### Example 1

99.2 g (0.5 mol) of dibenzyl ether, 120.0 g (2.0 mol) of acetic acid and 1.0 g of a sulfated silica gel (15% SO₃/l SiO₂) were heated at 120° C. in a flask with baffle and paddle stirrer with vigorous stirring (250 rpm) and under nitrogen. After a reaction time of 7 hours, the mixture was rapidly cooled, and the organic phase, after the addition of toluene and water, was separated off and analyzed by means of gas chromatography. The reaction mixture comprised benzyl acetate and dibenzyl ether in the ratio 30:65.

### Example 2

Example 2 was carried out analogously to Example 1. 300.3 g (5.0 mol) of acetic acid and 3.0 g of a sulfated alumosilicate (15% SO₃/l Al₂O₃.SiO₂) were used. The reaction time was 7 hours. The reaction mixture comprised benzyl acetate and dibenzyl ether in the ratio 42:54.

### Example 3

Example 3 was carried out analogously to Example 1. 300.3 g (5.0 mol) of acetic acid and 3.0 g of a sulfated silica gel (15% SO₃/l SiO₂) were used. The reaction time was 7 hours. The reaction mixture comprised benzyl acetate and dibenzyl ether in the ratio 53:41.

### Example 4

Example 4 was carried out analogously to Example 1. 300.3 g (5.0 mol) of acetic acid and 3.0 g of a sulfated silica gel (41% SO₃/l SiO₂) was used. The reaction time was 5 hours. The reaction mixture comprised benzyl acetate and dibenzyl ether in the ratio 70:22.

### Example 5

Example 1 was repeated, but using 30.0 g (0.5 mol) of acetic acid, 51.0 g (0.5 mol) of acetic anhydride and 3.0 g of a sulfated niobium oxide (75 g SO₃/l Nb₂O₅) at 100° C. The reaction time was 7 hours. The reaction mixture comprised benzyl acetate and dibenzyl ether in the ratio 45:42.

### Example 6

Example 5 was repeated but using 3.0 g of a sulfated aluminium oxide SPH 501 from Rhone-Poulenc (15% SO₃/l Al₂O₃). The reaction time was 5 hours. The reaction mixture comprised benzyl acetate and dibenzyl ether in the ratio 63:26.

### Example 7

Example 5 was repeated, but using 3.0 g of a sulfated calcium sulfate (17% SO₃/l CaSO₄). The reaction time was 1 hour. The reaction mixture comprised benzyl acetate and dibenzyl ether in the ratio 75:12.

### Example 8

Example 5 was repeated, but using 3.0 g of a sulfated alumosilicate (15% SO₃/l Al₂O₃.SiO₂). The reaction time was 3 hours. The reaction mixture comprised benzyl acetate and dibenzyl ether in the ratio 89:3.

### Example 9

Example 5 was repeated, but using 3.0 g of a sulfated aluminium oxide SPH 501 from Rhone-Poulenc (15% SO₃/l Al₂O₃). The reaction time was 5 hours. The reaction mixture comprised benzyl acetate and dibenzyl ether in the ratio 63:26.

### Example 10

Example 5 was repeated, but using 3.0 g of a sulfated tantalum oxide (15% SO₃/l Ta₂O₅). The reaction time was 5 hours. The reaction mixture comprised benzyl acetate and dibenzyl ether in the ratio 86:5.

### Example 11

Example 5 was repeated, but using 3.0 g of a sulfated silica gel (41% SO₃/l SiO₂). The reaction time was 15 minutes. The reaction mixture comprised benzyl acetate and dibenzyl ether in the ratio 86:5.

### Example 12

Example 5 was repeated, but using 0.5 g of a sulphated titanium oxide (Aldrich). The reaction time was 7 hours. The reaction mixture comprised benzyl acetate and dibenzyl ether in the ratio 51:35.

### Example 13

Example 5 was repeated, but using 0.5 g of a sulfated zirconium oxide (Acros). The reaction time was 5 hours. The reaction mixture comprised benzyl acetate and dibenzyl ether in the ratio 71:14.

### Example 14

Example 5 was repeated, but using 3.0 g of a catalyst prepared by applying 20 g of trifluoromethanesulfonic acid to 1000 ml of silica gel. The reaction time was 3 hours. The reaction mixture comprised benzyl acetate and dibenzyl ether in the ratio 83:5.

### Example 15

Example 5 was repeated, but using 37.0 g (0.5 mol) of propionic acid, 65.1 g (0.5 mol) of propionic anhydride and 3.0 g of a sulfated silica gel (15% SO₃/l SiO₂). The reaction time was 7 hours. The reaction mixture comprised benzyl propionate and dibenzyl ether in the ratio 76:12.

### Example 16

### Work-Up

Example 5 was repeated, but using 3.0 g of a sulfated silica gel (41% SO₃/l SiO₂), and worked up after a running time of 7 hours. Following filtration and distillative separation of the reaction mixture, 106 g (70%) of benzyl acetate with a purity of 97.5% are isolated at 57-60° C./0.25 mbar. Forerunnings and after-runnings comprise a further 7.4 g (5%) of benzyl acetate.

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

What is claimed is:

1. A process for preparing a benzyl carboxylate of the formula 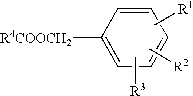
wherein
R¹ to R³ are identical or different and are C₁-C₆-alkyl, C₁-C₆-alkoxy, C₁-C₆-haloalkyl, C₁-C₆-haloalkoxy, CN, CO(C₁-C₆-alkyl), NO₂ or halogen and
R⁴ is hydrogen, C₁-C₆-alkyl, C₁-C₆-alkenyl, C₁-C₁₂-aryl, C₁-C₆-haloalkyl, C₁-C₆-haloalkenyl or C₁-C₁₂-haloaryl,
from dibenzyl ethers,
the process comprising reacting (A) dibenzyl ethers of the formula 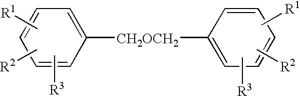
wherein R¹, R² and R³ have the meanings given above,
or mixtures of dibenzyl ethers and benzyl alcohols of the formula 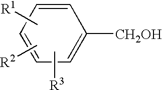
wherein R¹, R² and R³ have the meanings given above
with (B) carboxylic acids of the formula
R⁴COOH
wherein R⁴ has the meaning given above,
in the presence of a catalyst comprising at least one acid, wherein the acid is on a support.

2. The process according to claim 1, wherein the acid is a component selected from the group consisting of inorganic acids, organic acids having a pH of from 1 to 6, Lewis acids having a pH of from 1 to 6, and mixtures thereof.

3. The process according to claim 1, wherein the acid is a component selected from the group consisting of sulfur trioxide, sulfuric acid, hydrogen chloride, hydrogen bromide, hydrogen iodide, hydrofluoric acid, perchloric acid, chlorosulfonic acid, phosphoric acid, trifluoroacetic acid, methanesulfonic acid, ethanesulfonic acid, benzenesulfonic acid, 4-toluenesulfonic acid, boron trifluoride, aluminium chloride, aluminium bromide, aluminium iodide, zinc chloride, tin chloride, titanium chloride, zirconium chloride, and mixtures thereof.

4. The process of claim 3, wherein the acid is applied to more than one support.

5. The process according to claim 1, wherein the support is a component selected from the group consisting of oxides of elements of group II A, group III B, group IV B, group VB, group VII B, group VIII, group IIIA, and group IVA, sulfates of elements of group II A, group III B, group IV B, group VB, group VII B, group VIII, group IIIA, group IVA, and mixtures thereof.

6. The process according to claim 1, wherein the catalyst is a component selected from the group consisting of sulfur trioxide, sulfuric acid, trifluoromethanesulfonic acid, CaO, MgO, ZrO₂, TiO₂, HfO₂, SnO₂, Al₂O₃, SiO₂, Al₂O₃.SiO₂, alumosilicates, Nb₂O₅, Ta₂O₅, Fe₂O₃, LaSO₄, CaSO₄, and mixtures thereof

7. The process according to claim 6, wherein the support comprises activated carbon.

8. The process according to claim 1, wherein the catalyst is a sulfated metal oxide.

9. The process according to claim 1, wherein the dibenzyl ether is unsubstituted dibenzyl ether.

10. The process according to claim 1, wherein the dibenzyl ether is a substituted dibenzyl ether which carries one or more substituents from the series C₁-C₆-alkyl, C₁-C₆-alkoxy, CN, CO(C₁-C₆-alkyl), NO₂ or halogen.

11. The process according to claim 1, wherein 2 to 50 equivalents of carboxylic acid, based on dibenzyl ether, are used.

12. The process according to claim 1, wherein the reaction takes place in the presence of dehydrating agents.

13. The process according to claim 1, wherein the reaction takes place with the removal of water by distillation or by passing nitrogen through.

14. The process according to claim 1, wherein the reaction is carried out in the presence of the corresponding anhydride of the carboxylic acid used.

15. The process according to claim 12, wherein anhydride is from about 0.1 to about 10 equivalents of, based on dibenzyl ether.

16. The process according to claim 1, wherein the reaction is carried out at a temperature of from 15 to 200° C.

17. The process according to claim 1, wherein one or more supported acids are used in an amount of from 0.5 to 100% by weight, based on the amount of dibenzyl ether, in the case of a suspended catalyst, or with space velocities of from 1.0 to 3000 g of dibenzyl ether per liter of heterogenized superacid per hour when the catalyst is arranged as a fixed bed.
